SEQUENCE LISTING (21 sequences)

SEQ ID NO: 1 - DNA, 28 bp, Artificial Sequence
  primer oligonucleotide
taagtatgttgaagaaagattattgtag

SEQ ID NO: 2 - DNA, 22 bp, Artificial Sequence
  primer oligonucleotide
cgcatcaactaaatcattaaaa

SEQ ID NO: 3 - DNA, 18 bp, Artificial Sequence
  oligonucleotide
tataaacacgtctttcna

SEQ ID NO: 4 - DNA, 18 bp, Artificial Sequence
  oligonucleotide
tataaacacatctttcna

SEQ ID NO: 5 - DNA, 18 bp, Artificial Sequence
  oligonucleotide
tataaacacgtctttcaa

SEQ ID NO: 6 - DNA, 18 bp, Artificial Sequence
  oligonucleotide
tataaacacatctttcaa

SEQ ID NO: 7 - DNA, 18 bp, Artificial Sequence
  oligonucleotide
tataaacacgtctttcaa

SEQ ID NO: 8 - DNA, 18 bp, Artificial Sequence
  oligonucleotide
tataaacacatctttcaa

SEQ ID NO: 9 - DNA, 19 bp, Artificial Sequence
  oligonucleotide
tnataaacacgtctttcaa

SEQ ID NO: 10 - DNA, 19 bp, Artificial Sequence
  oligonucleotide
tnataaacacatctttcaa

SEQ ID NO: 11 - DNA, 18 bp, Artificial Sequence
  oligonucleotide
ttcaacttatataaacnc

SEQ ID NO: 12 - DNA, 19 bp, Artificial Sequence
  oligonucleotide
tntctttcaaaattcacat

SEQ ID NO: 13 - DNA, 19 bp, Artificial Sequence
  oligonucleotide
gntctttcaaaattcacat

SEQ ID NO: 14 - DNA, 18 bp, Artificial Sequence
  oligonucleotide
ttcaacttatataaacnc

SEQ ID NO: 15 - DNA, 19 bp, Artificial Sequence
  oligonucleotide
atnctttcaaaattcacat

SEQ ID NO: 16 - DNA, 19 bp, Artificial Sequence
  oligonucleotide
gntctttcaaaattcacat

SEQ ID NO: 17 - DNA, 18 bp, Artificial Sequence
  oligonucleotide
ttcaacttatataaacac

SEQ ID NO: 18 - DNA, 19 bp, Artificial Sequence
  oligonucleotide
catctttcaaaattcacat

SEQ ID NO: 19 - DNA, 19 bp, Artificial Sequence
  oligonucleotide
cgtctttcaaaattcacat

SEQ ID NO: 20 - DNA, 28 bp, Artificial Sequence
  primer oligonucleotide
taagtatgttgaagaaagattattgtag

SEQ ID NO: 21 - DNA, 22 bp, Artificial Sequence
  primer oligonucleotide
cgcatcaactaaatcattaaaa

(19) United States
(12) Patent Application Publication
(10) Pub. No.: US 2006/0166201 A1
(43) Pub. Date: Jul. 27, 2006

(54) Method for the detection of nucleic acid sequences by means of crackable probe molecules

(76) Inventors: Philipp Schatz, Berlin (DE); Matthias Schuster, Berlin (DE); Kurt Berlin, Stahnsdorf (DE)

Correspondence Address: KRIEGSMAN & KRIEGSMAN, 30 TURNPIKE ROAD, SUITE 9, SOUTHBOROUGH, MA, 01772, US

(21) Appl. No.: 10/526,277

(22) PCT Filed: Sep. 1, 2003
(86) PCT No.: PCT/DE03/02936

§ 371 Date: Sep. 19, 2005

(30) Foreign Application Priority Data
Sep. 1, 2002 (DE) 102407460

(51) Int. Cl. C12Q 1/68

(52) U.S. Cl. 435/6

(57) ABSTRACT

The present invention describes a method for the detection of nucleic acid sequences, which is characterized in that the following steps are conducted: a) at least one nucleic acid is bound to a solid phase; b) probe molecules are hybridized to the nucleic acids in a sequence-specific manner, whereby the probe molecules are provided with a cleavable bond and a mass label, which is specific for the probe molecule; c) removal of the unhybridized probe molecules; d) contacting of the hybridized probe molecules with a matrix, which cleaves said cleavable bonds and at the same time serves as the matrix in a MALDI mass spectrometer; e) detection of the mass label at those positions where the nucleic acid was bound.

The present invention concerns a method for the detection of nucleic acid sequences in nucleic acids.

The levels of observation that have been well studied in molecular biology according to developments in methods in recent years include the genes themselves, the transcription of these genes into RNA and the translation to proteins therefrom. During the course of development of an individual, which gene is turned on and how the activation and inhibition of certain genes in certain cells and tissues are controlled can be correlated with the extent and nature of the methylation of the genes or of the genome.

5-Methylcytosine is the most frequent covalently modified base in the DNA of eukaryotic cells. For example, it plays a role in the regulation of transcription, in genetic imprinting and in tumorigenesis. The identification of 5-methylcytosine as a component of genetic information is thus of considerable interest. 5-Methylcytosine positions, however, cannot be identified by sequencing, since 5-methylcytosine has the same base-pairing behavior as cytosine. In addition, in the case of a PCR amplification, the epigenetic information which is borne by the 5-methylcytosines is completely lost.

A relatively new method that in the meantime has become the most frequently used method for investigating DNA for 5-methylcytosine is based on the specific reaction of bisulfite with cytosine, which, after subsequent alkaline hydrolysis, is converted to uracil, which corresponds in its base-pairing behavior to thymidine. In contrast, 5-methylcytosine is not modified under these conditions. Thus, the original DNA is converted so that methylcytosine, which originally cannot be distinguished from cytosine by its hybridization behavior, can now be detected by “standard” molecular biology techniques as the only remaining cytosine, for example, by amplification and hybridization or sequencing. All of these techniques are based on base pairing, which is now fully utilized. The prior art, which concerns sensitivity, is defined by a method that incorporates the DNA to be investigated in an agarose matrix, so that the diffusion and renaturation of the DNA is prevented (bisulfite reacts only on single-stranded DNA) and all precipitation and purification steps are replaced by rapid dialysis (Olek A, Oswald J, Walter J. A modified and improved method for bisulphite based cytosine methylation analysis. Nucleic Acids Res. Dec. 15, 1996; 24(24):5064-6). Individual cells can be investigated by this method, which illustrates the potential of the method. Of course, up until now, only individual regions of up to approximately 3000 base pairs long have been investigated; a global investigation of cells for thousands of possible methylation analyses is not possible. Of course, this method also cannot reliably analyze very small fragments from small quantities of sample. These are lost despite the protection from diffusion through the matrix.

An overview of other known possibilities for detecting 5-methylcytosines can be derived from the following review article: Rein T, DePamphilis M L, Zorbas H. Identifying 5-methylcytosine and related modifications in DNA genomes. Nucleic Acids Res. May 15, 1998; 26(10):2255-64.

The bisulfite technique has been previously applied only in research, with a few exceptions (e.g., Zeschnigk M, Lich C, Buiting K, Dörfler W, Horsthemke B. A single-tube PCR test for the diagnosis of Angelman and Prader-Willi syndrome based on allelic methylation differences at the SNRPN locus. Eur J Hum Genet. March-April 1997; 5(2):94-8). However, short, specific segments of a known gene have always been amplified after a bisulfite treatment and either completely sequenced (Olek A, Walter J. The pre-implantation ontogeny of the H19 methylation imprint. Nat Genet. November 1997; 17(3):275-6) or individual cytosine positions have been detected by a “primer extension reaction” (Gonzalgo M L, Jones P A. Rapid quantitation of methylation differences at specific sites using methylation-sensitive single nucleotide primer extension (Ms-SNuPE). Nucleic Acids Res. Jun. 15, 1997; 25(12):2529-31, WO-A 95/00669) or an enzyme step (Xiong Z, Laird P W. COBRA: a sensitive and quantitative DNA methylation assay. Nucleic Acids Res. Jun. 15, 1997; 25(12):2532-4). Detection by hybridization has also been described (Olek et al., WO-A 99/28498).

A newer method is also the detection of cytosine methylation by means of a Taqman PCR, which has become known as “methyl light” (WO-A 00/70090). With this method, it is possible to detect the methylation status of individual positions or a few positions directly in the course of the PCR, so that a subsequent analysis of the products becomes superfluous.

Genomic DNA is obtained from DNA of cells, tissue or other assay samples by standard methods. This standard methodology is found in references such as Fritsch and Maniatis, Molecular Cloning: A Laboratory Manual, 1989.

A plurality of mass-labeled oligonucleotides, which are simple to produce and do not fragment, have been used for labeling amplificates (www.quiagengenomics.com).

For example, trityl groups with different masses are used as mass labels (Shchepinov, M. S., Southern E. M. Trityl mass-tags for encoding in combinatorial oligonucleotide synthesis (1999), Nucleic Acids Symposium Series 42: 107-108).

Matrix-assisted laser desorption/ionization mass spectrometry (MALDI-TOF) is a very powerful development for the analysis of biomolecules (Karas M, Hillenkamp F. Laser desorption ionization of proteins with molecular masses exceeding 10,000 daltons. Anal Chem. Oct. 15, 1998; 60(20):2299-301). An analyte is embedded in a light-absorbing matrix. The matrix is vaporized by a short laser pulse and the analyte molecule is transported unfragmented into the gaseous phase. The analyte is ionized by collisions with matrix molecules. An applied voltage accelerates the ions in a field-free flight tube. Ions are accelerated to varying degrees based on their different masses. Smaller ions reach the detector sooner than large ions. The time of flight is converted to the mass of the ions.

Technical innovations in hardware have significantly improved the method. In this regard, the “delayed extraction” (DE) method should be mentioned. For DE, the acceleration voltage is turned on with a delay relative to the laser pulse and in this way, an improved resolution of the signals is achieved, since the number of collisions is reduced.

MALDI-TOF spectroscopy is excellently suitable for the analysis of peptides and proteins. The analysis of nucleic acids is somewhat more difficult (Gut, I. G. and Beck, S. (1995), DNA and Matrix Assisted Laser Desorption Ionization Mass Spectrometry. Molecular Biology: Current Innovations and Future Trends 1: 147-157.) For nucleic acids, the sensitivity is approximately 100 times poorer than for peptides and decreases overproportionally with increasing fragment size. For nucleic acids, which have a backbone with a plurality of negative charges, the ionization process through the matrix is basically inefficient. In MALDI-TOF spectroscopy, the choice of the matrix plays an imminently important role. Several very powerful matrixes, which produce a very fine crystallization, have been found for the desorption of peptides. In the meantime, several effective matrixes have also been developed for DNA, but the difference in sensitivity has not been reduced thereby. The difference in sensitivity can be reduced by modifying the DNA chemically in such a way that it resembles a peptide.

Phosphorothioate nucleic acids, in which the usual phosphates of the backbone are substituted by thiophosphates, can be converted by simple alkylation chemistry into a charge-neutral DNA (Gut, I. G. und Beck, S. (1995), A procedure for selective DNA alkylation and detection by mass spectrometry. Nucleic Acids Res. 23: 1367-1373). The coupling of a “charge tag” to this modified DNA results in an increase in sensitivity of the same magnitude as is found for peptides. Another advantage of “charge tagging” is the increased stability of the analysis in the presence of impurities, which make the detection of unmodified substrates very difficult. PNAs and methylphosphonate oligonucleotides have been investigated with MALDI and can be analyzed in this way.

At the present time, this technology can distinguish molecules with a mass difference of 1 Da, in the mass range of 1,000 to 4,000 Da. Due to the natural distribution of isotopes, most biomolecules, however, vary within approximately 5 Da. Technically, this mass spectrometric method is thus especially suitable for the analysis of biomolecules. More reasonably, the products to be analyzed and which are to be distinguished in this way must be at least 5 Da apart. Therefore, 600 molecules could be distinguished in this mass range.

As probe molecules, PNA and LNA have been described many times in addition to DNA in the literature. PNA involves a synthetic nucleic acid analog, where the sugar-phosphate backbone is replaced by a polyamide similar to a peptide. PNAs have N and C ends instead of 5′ and 3′ ends. Like LNAs (Locked Nucleic Acids) (see www.cureon.com/technology/aboutlna), PNAs provide a high stability against nucleases and a high binding affinity to complementary DNA.

Photocleavable units, which permit a light-controlled release of samples, are described for MALDI-TOF measurements (Olejnik et al., 1998, Nucleic Acids Res., 3572-3576). Koster et al. (WO-A 98/20166) proposed the use of cleavable compounds. For this purpose, primer oligonucleotides were first immobilized and then were hybridized with genomic DNA. After a subsequent extension reaction, the products that formed were specifically cleaved from the surface and analyzed by mass spectrometry. In a similar manner, the use of photolytically cleavable oligonucleotide probes on an array was proposed (Jäschke, A., Hausch, F. EP 1,138,782), wherein a multiplex sequence-dependent modification of the oligonucleotide probes is conducted and the masses of the modified probes are measured directly on the array. The mixture of target sequences is thus separated by the defined positions of the probes on the array.

Matrix-induced fragmentation of DNA containing P3′-N5′ phosphoramidate is described in the literature (Shchepinov, M., Denissenko, M., 2001, Nucleic Acids Res., 3864-3872). In this way, the P—N bond can be cleaved under acidic conditions.

### PRESENTATION OF THE PROBLEM

The object of the present invention is to provide a method for the detection of nucleic acid sequences. For this purpose, probe molecules will be hybridized in a sequence-specific manner to one or more nucleic acids immobilized on solid phases. These probe molecules will be provided with a cleavable bond and a specific mass label. Then the hybridized probe molecules will be contacted with a substance or a substance mixture, which cleaves the cleavable bonds and also serves as the matrix in a MALDI mass spectrometer. The mass labels will then be detected at the positions on the solid phase, at which the nucleic acids were bound.

### DESCRIPTION OF THE INVENTION

A method is described for the detection of nucleic acid sequences. This method is characterized by the following steps:

In the first step of the method, at least one nucleic acid sample is bound to a solid phase. In the next step, the probe molecules are hybridized in a sequence-specific manner to the nucleic acid sample, whereby the probe molecules are provided with a cleavable bond and a mass label, which is specific for the probe molecule. Then the unhybridized probe molecules are removed. In a further step of the method, the hybridized probe molecules are contacted by a matrix, which cleaves the cleavable bonds and also serves as the matrix in a MALDI mass spectrometer. The mass labels are detected in the last step of the method at those positions where the nucleic acid sample was bound.

This method is described in detail in the following:

A nucleic acid is obtained preferably from cell lines, blood, sputum, stool, urine, cerebrospinal fluid, tissue embedded in paraffin (for example, tissue from eyes, intestine, kidney, brain, heart, prostate, lung, breast or liver), histological slides or all possible combinations thereof.

The nucleic acid is amplified, whereby this amplification is preferably produced by means of enzymatic primer extension, PCR, rolling circle amplification, ligase chain reaction or another method.

In a particularly preferred variant of the method, the amplification of several different fragments is conducted in one reaction vessel.

At least one nucleic acid is bound to a solid phase, which can preferably also serve as a sample support for a mass spectrometer. The solid phase or the surface of the solid phase most preferably consists of nonconducting materials, such as glass, for example.

Particularly preferred also are conducting materials, such as, for example, teflon, silicon or black conductive polypropylene. Other materials such as polystyrene, aluminum, steel, iron, copper, nickel, silver, or gold are also preferred.

According to the invention, the binding of nucleic acids to the surface can be accomplished both covalently (e.g., by a primer which bears a thiol at the 5′-end and is bound to a surface activated with bromoacetic acid) as well as noncovalently by van der Waals forces or hydrogen bridges (e.g., by heat immobilization or incubation).

Preferably, several nucleic acids are disposed on a solid phase surface in the form of a rectangular or hexagonal grid. Alternatively, it is preferred that each time at least one nucleic acid is disposed on a plurality of surfaces. These solid phases can be utilized most preferably in the sample support of a MALDI mass spectrometer.

The nucleic acids to be detected preferably involve DNA sequences and particularly sequences that are variable among different samples and that contain SNPs, point mutations, deletions, inversions or insertions. The nucleic acid sequences to be detected are most preferably chemically pretreated DNA sequences, which serve for the detection of DNA methylation at specific CpG positions.

The chemical treatment is most preferably conducted with a bisulfite (=disulfite, hydrogen sulfite). In this way, all cytosines that are not present in the CpG context are converted to thymidine, whereupon the investigation of individual CpG positions is made possible.

Preferably, the chemical treatment is conducted after embedding the DNA in agarose. It is also preferred that in the chemical treatment, a reagent that denatures the DNA duplex and/or a radical trap is/are present.

The probe molecules necessary for binding to the nucleic acids to be detected are produced in the prior art often combinatorially in the form of libraries (EP 1,036,202), which also preferably find application in the method according to the invention.

Probe molecules are most preferably provided with a cleavable bond and a mass label, which is specific for the repective probe molecule. Such labels can be, for example, 6-triethylammoniumhexyryl, 6-trimethylammoniumhexyryl or acid-labile monomethoxytrityl or 4-methyltrityl protective groups.

The mass of a label preferably differs each time by at least 1 Da from the masses of all other labels used in one experiment. The probe molecules preferably comprise at least one CG, TG or CA dinucleotide.

The different mass can most preferably also be the result of an enzymatic reaction. In this case, the probe is synthesized with a mass label and is then modified enzymatically, whereby the mass changes.

The mass label is most preferably first combined with the probe molecule as a consequence of an enzymatic modification. In this case, the probe is produced chemically without a mass label and then enzymatically provided with a mass label.

Preferably also, the probe is provided chemically with a mass label and is not enzymatically modified. In addition, it is preferred according to the invention that the probe provided with a mass label is chemically modified. This can be accomplished, for example, by acids or with a treatment according to Maxam and Gilbert.

These probe molecules, which most preferably consist of DNA or modified DNA, are hybridized in a sequence-specific manner to the nucleic acids. Preferably also, the probe molecules are RNA, LNA, PNA or corresponding hybrids thereof, also combined with DNA or modified DNA. The unhybridized probe molecules are removed. After hybridization, the remaining hybridized probe molecules are preferably modified enzymatically by primer extension or ligation. As enzymes for the primer extension, for example, thermosequenase or Taq polymerase are considered, whereas for the ligation, for example, Ampligase DNA ligase, Pfu or Taq DNA ligase find application.

The hybridization of the amplificates is preferably performed with two classes of probe molecules, each with at least one member, whereby the probe molecules of the first class preferably hybridize to the sequence which arises from the chemical treatment of the genomic DNA, if a cytosine to be investigated was present in the methylated state in the genomic DNA and whereby the probe molecules of the second class preferably hybridize to the sequence which arises from the chemical treatment of the genomic DNA, if a cytosine to be investigated was present in the unmethylated state in the genomic DNA.

A hybridization is preferably performed with two classes of probe molecules, each with at least one member, whereby the probe molecules of the first class preferably hybridize to the sequence which arises from the chemical treatment of the genomic DNA, if a cytosine to be investigated was present in the methylated state in the genomic DNA and less preferably to the sequence which arises from the chemical treatment of the genomic DNA, if a cytosine to be investigated was present in unmethylated state in the genomic DNA and whereby the oligomers of the second class hybridize to the amplificate to be investigated essentially independently of the methylation of said specific cytosine in the genomic DNA.

A hybridization is preferably performed with two classes of probe molecules, each with at least one member, whereby the probe molecules of the first class preferably hybridize to the sequence which arises from the chemical treatment of the genomic DNA, if a cytosine to be investigated was present in the unmethylated state in the genomic DNA and less preferably to the sequence which arises from the chemical treatment of the genomic DNA, if a cytosine to be investigated was present in methylated state in the genomic DNA and whereby the oligomers of the second class hybridize to the amplificate to be investigated essentially independently of the methylation of said specific cytosine in the genomic DNA. Then the unhybridized probe molecules are removed.

The hybridized probe molecules are contacted with a matrix (e.g., by spraying, pipetting, spotting), which cleaves the cleavable bonds and at the same time serves as the matrix in a MALDI mass spectrometer. For this purpose, for example, a 2′,4′,6′-trihydroxyacetophenone (THA) matrix or a 3-HPA (3-hydroxypicolinic acid) matrix is considered, wherein the THA matrix is reacted with dilute acid such as TFA (trifluoroacetic acid) according to the invention. The detection limit can be decisively reduced due to the size of the oligonucleotide cleavage product. Examples of acid-cleavable protective groups are monomethoxytrityl or 4-methyltrityl.

The oligonucleotides can also be cleaved in a structure-specific manner by addition of a flap endonuclease such as Cleavase VIII. Further, endonucleases can be utilized for the cleavage, which cleave the probe from the center, such as, for example, mung bean nuclease or T7 endonuclease I. In addition, the use of sequence-specific endonucleases for cleavage is possible, for example, the enzymes Tsp 509I or MseI can be utilized for this purpose. Also, digestion with a 3′-endonuclease is possible, preferably after addition of an acidic matrix. Further, exonucleases are utilized for the cleavage. A 3′,5′-exonuclease, such as, for example, exonuclease I cleaves the single-stranded probe from the 3′-end up to a modification (e.g., phosphothioate). A 5′,3′-exonuclease, such as, for example, T7 exonuclease correspondly cleaves the single-stranded probe from the 5′-end up to a modification.

The mass labels are detected at those positions where the nucleic acid was bound. This detection is most preferably produced by means of MALDI-TOF mass spectrometry. The detection limit is decisively reduced due to the preferred charge of a single positive charge or a single negative charge for the mass label.

The above-described method is preferably used for the diagnosis and/or prognosis of adverse events for patients or individuals, whereby these adverse events belong to at least one of the following categories: undesired drug effects; cancer diseases; CNS malfunctions, damage or disease; symptoms of aggression or behavioral disturbances; clinical, psychological and social consequences of brain damage; psychotic disturbances and personality disorders; dementia and/or associated syndromes; cardiovascular disease, malfunction and damage; malfunction, damage or disease of the gastrointestinal tract; malfunction, damage or disease of the respiratory system; lesion, inflammation, infection, immunity and/or convalescence; malfunction, damage or disease of the body as [a consequence of] an abnormality in the development process; malfunction, damage or disease of the skin, the muscles, the connective tissue or the bones; endocrine and metabolic malfunction, damage or disease; headaches or sexual malfunction.

The above-described method is preferably used for distinguishing cell types or tissues or for investigating cell differentiation.

A kit, comprising a solid phase for immobilizing nucleic acids, probe molecules, components for conducting the mass-spectrometric measurement as well as instructions for conducting the method, is also preferred.

The Following Examples Explain the Method According to the Invention:

Conducting the methylation analysis in the MDR1 gene by means of a cleavable probe

### EXAMPLE

### Binding of the Target Sequence to the Solid Phase

In the first step, a genomic sequence is treated with the use of bisulfite (hydrogen sulfite, disulfite) in such a way that all of the unmethylated cytosines at the 5-position of the base are modified such that a base that is different in its base-pairing behavior is formed, while the cytosines that are methylated in the 5-position remain unchanged. If bisulfite is used for the reaction, then an addition occurs on the unmethylated cytosine bases. Also, a denaturing reagent or solvent as well as a radical trap must be present. A subsequent alkaline hydrolysis then leads to the conversion of unmethylated cytosine nucleobases to uracil. This converted DNA serves for the detection of methylated cytosines. In the second step of the method, the treated nucleic acids are diluted with water or an aqueous solution. Preferably, a desulfonation of the DNA is then conducted.* In the third step of the method, the nucleic acid is amplified in a polymerase chain reaction, preferably with a heat-stable DNA polymerase. The PCR reactions were conducted in a thermocycler (Eppendorf GmbH). For a 100 μl batch, 40 ng of DNA, 0.07 μmol/l of each primer oligonucleotide, 1 mmol/l dNTPs and four units of HotstarTaq were utilized. The other conditions were selected according to the manufacturer's instructions. For the PCR, first a denaturation was conducted for 15 minutes at 96° C., then 40 cycles (60 seconds at 96° C., 75 seconds at 56° C. and 75 seconds at 65° C.) and then a [dilution] in E1 water or an aqueous solution. Preferably, a desulfonation of the DNA is then conducted. In the third step of the method, the nucleic acid is amplified in a polymerase chain reaction, preferably with a heat-stable DNA polymerase. The PCR reactions were conducted in a thermocycler (Eppendorf GmbH). For a 100 μl batch, 40 ng of DNA, 0.07 μmol/l of each primer oligonucleotide, 1 mM dNTPs and four units of HotstarTaq were utilized. The other conditions were selected according to the manufacturer's instructions. For the PCR, first a denaturation was conducted for 15 minutes at 96° C., then 40 cycles (60 seconds at 96° C., 75 seconds at 56° C. and 75 seconds at 65° C.) and a subsequent elongation of 10 minutes at 72° C. The presence of the PCR products was confirmed on agarose gels. One of the two primer oligonucleotides was modified at its thiol 5′-end (in the following Example 8, the phosphate 5′-end instead.

In the present case, cytosines from the potential promotor region of the MDR1 gene are investigated. The reaction of a patient to chemotherapy can be followed with sequences of this gene. For this purpose, a defined fragment of 242 bp length is amplified with the specific primer oligonucleotides SH-TAA GTA TGT TGA AGA AAG ATT ATT GTA G (Seq. ID 1) and CGC ATC AAC TAA ATC ATT AAA A (Seq. ID 2). This amplificate is bound by its thiol modification to a polylysine solid phase treated with bromoacetic acid. Polylysine-coated glass slides were cleaned by ultrasound prior to this and activated for 1 h in a solution of 20 mmol/l bromoacetic acid, 20 mmol/l dicylohexylcarbodiimide, and 2 mmol/l 4-(dimethylamino)pyridine. The binding with the PCR product is produced in a buffer solution of 0.18 mol/l Tris-carboxyethylphosphine and 200 mmol/l NaH₂PO₄ in a moist chamber warmed to 25° C. The PCR products are denatured with 0.05 mol/l NaOH and then analyzed.

### Example 1

The single-stranded PCR product was hybridized with an oligonucleotide probe and formed a duplex structure. For this purpose, acid-labile modified oligonucleotides were used: 5′-TAT AAA CAC GTC TTT CApnA-amino-3′ (Seq. ID 3) or 5 -TAT AAA CAC ATC TTT CapnA-amino-3′ (Seq. ID 4), wherein the cytosine to be detected is found at position 198 of the amplificate. The adenosine at the next-to-last position of both oligonucleotides involves a 5′-amino-adenosine, which is readily hydrolyzed by acid. A 6-triethylammoniumhexyryl-N-hydroxysuccinimidyl ester (199 Da) (CT1), or a 6-trimethylammoniumhexyryl-N-hydroxysuccinimidyl ester (129 Da) (CT2) is coupled beforehand to the amino function at the 3′-end. In this way, the masses of the two smaller cleavage products differ by about 70 Da. The methylated cytosine is detected with the oligonucleotide (Seq. ID 3), while, on the other hand, the unmethylated state, which is represented by a thymine, is detected with the oligonucleotide (Seq. ID 4). Both oligonucleotides hybridize to the complementary strand each time. The acid-labile cleavage site is hydrolyzed by introducing 350 mmol/l 3-HPA in acetonitrile containing 1.5% trifluoroacetic acid. The detection of the hybridization product is based on the detection of the mass of the cleavage products by means of MALDI-TOF mass spectrometry. The detection limit is decisively reduced due to the size of the oligonucleotide cleavage product and the defined single positive charge. A hybridization reaction of the amplified DNA with the probe (Seq. ID 3, Seq. ID 4) occurs only if a methylated cytosine was present at this site in the bisulfite-treated DNA. Thus the methylation status of the respective cytosine to be investigated decides the hybridization product and thus the detected mass. By incorporating the PN bond directly at the 3′-end, the additional advantage of being able to use an uncharged mass label is obtained. For example, a peptide can then be coupled.

### Example 2

The single-stranded PCR product was hybridized with an oligonucleotide probe and formed a duplex structure. For this purpose, modified oligonucleotides were used: 5′-amino-TAT AAA CAC GTC TTT CAA (Seq. ID 5) or 5′-amino-TAT AAA CAC ATC TTT CAA (Seq. ID 6). A 6-triethylammoniumhexyryl-N-hydroxysuccinimidyl ester (199 Da) (CT1), or a 6-trimethylammoniumhexyryl-N-hydroxysuccinimidyl ester (129 Da) (CT2) is coupled beforehand to the amino function at the 5′-end. In this way, the masses of the two smaller cleavage products differ by about 70 Da. The methylated cytosine is detected with the oligonucleotide (Seq. ID 5), while, on the other hand, the unmethylated state, which is represented by a thymine, is detected with the oligonucleotide (Seq. ID 6). Both oligonucleotides hybridize to the complementary strand each time. The oligonucleotides are then subjected to a treatment according to Maxam and Gilbert. By introducing dimethyl sulfate and heating in alkaline pH, all adenosines and guanosines are cleaved. The detection of the hybridization product is based on the detection of the mass of the cleavage products by means of MALDI-TOF mass spectrometry. In this case, one observes a mass of (498 Da+199 Da(CT1+dT), or +129 Da(CT2+dT)) 697, or 627 Da. The detection limit can be decisively reduced due to the size of the oligonucleotide cleavage product. A hybridization reaction of the amplified DNA with the probe (Seq. ID 5, Seq. ID 6) occurs only if a methylated cytosine was present at this site in the bisulfite-treated DNA. Thus the methylation status of the respective cytosine to be investigated decides the hybridization product and thus the detected mass. If the cytosines and thymidines are cleaved with hydrazine and piperidine, a 3′-modified oligonucleotide pair can then be investigated. In this case, dAdA-CT1 and dAdA-CT2 result as cleavage products. All conceivable cleavage products, however, have multiple charges.

### Example 3

The single-stranded PCR product was hybridized with an oligonucleotide probe and formed a duplex structure. For this purpose, modified oligonucleotides were used: 5′-amino-TAT AAA CAC GTC TTT CAA (Seq. ID 7) or 5′-amino-5′-TAT AAA CAC ATC TTT CAA (Seq. ID 8). An acid-cleavable protective group such as 4-methyltrityl (258 Da), or monomethoxytrityl (289 Da) is coupled beforehand to the amino function at the 5′-end. In this way, the masses of the two smaller cleavage products differ by about 14 Da. The methylated cytosine is detected with the oligonucleotide (Seq. ID 7), while, on the other hand, the unmethylated state, which is represented by a thymine, is detected with the oligonucleotide (Seq. ID 8). Both oligonucleotides hybridize to the complementary strand each time. The oligonucleotides are covered over with an acid-containing 2′,4′,6′-trihydroxyacetophenone matrix and measured by MALDI-TOF. In this way, the oligonucleotides are separated by their mass labels. The detection of the hybridization product is based on the detection of the mass of the protective group by means of MALDI-TOF mass spectrometry. The detection limit is decisively reduced due to the size of the protective group and the defined charge of +1. A hybridization reaction of the amplified DNA with the probe (Seq. ID 7, Seq. ID 8) occurs only if a methylated cytosine was present at this site in the bisulfite-treated DNA. Thus the methylation status of the respective cytosine to be investigated decides the hybridization product and thus the detected mass.

### Example 4

The single-stranded PCR product was hybridized with an oligonucleotide probe and formed a duplex structure. For this purpose, modified oligonucleotides were used: 5′-amino-TmptAT AAA CAC GTC TTT CAA-3′ (Seq. ID 9) or 5′-amino-TmptAT AAA CAC ATC TTT CAA-3′ (Seq. ID 10). The mpt is a methyl phosphonate. A 6-triethylammoniumhexyryl-N-hydroxysuccinimidyl ester (199 Da) (CT1), or a 6-trimethylammoniumhexyryl-N-hydroxysuccinimidyl ester (129 Da) (CT2) is coupled beforehand to the amino function at the 5′-end. In this way, the masses of the two smaller cleavage products differ by about 70 Da. The methylated cytosine is detected with the oligonucleotide (Seq. ID 9), while, on the other hand, the unmethylated state, which is represented by a thymine, is detected with the oligonucleotide (Seq. ID 10). Both oligonucleotides hybridize to the complementary strand each time. The oligonucleotides are digested with a 3′-endoglycosidase. The detection of the hybridization product is based on the detection of the mass of the remaining dT and of the charge tag by means of MALDI-TOF mass spectrometry. The detection limit is decisively reduced due to the size of the mass label and the defined charge of −1. A hybridization reaction of the amplified DNA with the probe (Seq. ID 9, Seq. ID 10) occurs only if a methylated cytosine was present at this site in the bisulfite-treated DNA. Thus, the methylation status of the respective cytosine to be investigated decides the hybridization product and thus the detected mass. The methyl phosphonate can also be placed completely at the 3′-end of the oligonucleotide and then a single negatively charged molecule is also obtained without a charge tag.

### Example 5

The single-stranded PCR product was hybridized with two oligonucleotide probes following one another in the sequence and formed a duplex structure. For this purpose, modified oligonucleotides were used: 5′-TTC AAC TTA TAT AAA CAmtpC-3′ (Seq. ID 11) and 5′-TmtpTC TTT CAA AAT TCA CAT-3′ (Seq. ID 12) or 5′-GmtpTC TTT CAA AAT TCA CAT-3′ (Seq. ID 13). The abbreviation mtp stands for methyl phosphonate. The methylated cytosine is detected by the ligation of Seq. ID 11 and Seq. ID 12, while, on the other hand, the unmethylated state, which is represented by a thymine, is detected by the ligation of Seq. ID 11 and Seq. ID 13. Both oligonucleotides hybridize to the complementary strand each time. The oligonucleotides are digested by 3′-endoglycosidase and 5′-endoglycosidase. The detection of the ligation product is based on the detection of the mass of the remaining nucleotides (AmtpCp-TmtpC or AmtpCp-TmtpC) by means of MALDI-TOF mass spectrometry. The detection limit is decisively reduced due to the size of the products and the defined single negative charge. The mass can be shifted further due to additional methyl phosphonates.

### Example 6

The single-stranded PCR product was hybridized with two oligonucleotide probes following one another in the sequence and formed a duplex structure. For this purpose, modified oligonucleotides were used: 5′-TTC AAC TTA TAT AAA CApnC-3′ (Seq. ID 14) and 5′-ATmtpC TTT CAA AAT TCA CAT-3′ (Seq. ID 15) or 5′-GmtpTC TTT CAA AAT TCA CAT-3′ (Seq. ID 16). Here, the abbreviation mtp stands for methyl phosphonate. The methylated cytosine is detected by the ligation of Seq. ID 14 and Seq. ID 15, while, on the other hand, the unmethylated state, which is represented by a thymine, is detected by the ligation of Seq. ID 14 and Seq. ID 15. Both oligonucleotides hybridize to the complementary strand each time. The oligonucleotides are digested by addition of acidic 3-HPA (3-hydroxypicolinic acid) matrix containing 0.3% TFA (trifluoroacetic acid) and digestion with a 3′-endoglycosidase. The detection of the ligation product is based on the detection of the mass of the remaining nucleotides (NH₃⁺-Cp-Ap-TmtpC or NH₃⁺-Cp-Gp-TmtpC) by means of MALDI-TOF mass spectrometry. The detection limit is decisively reduced due to the size of the products and the defined single negative charge. Instead of the methyl phosphonate an NP (nitrogen-phosphorus) bond, thus a 3′-amino-guanoside or a 3′-amino-thymidine, can also be utilized in the second oligonucleotide at the second position at the 5′-end. An NH₃⁺-Cp-ANH₃⁺ is formed.

### Example 7

The single-stranded PCR product was hybridized with two oligonucleotide probes following one another in the sequence and formed a duplex structure. The two oligonucleotides overlap. For this purpose, modified oligonucleotides were used: 5′-TTC AAC TTA TAT AAA CAC-3′ (Seq. ID 17) and 5′-amino-CATC TTT CAA AAT TCA CAT-3′ (Seq. ID 18) or 5′-amino-CGTC TTT CAA AAT TCA CAT-3′ (Seq. ID 19). A 6-triethylammoniumhexyryl-N-hydroxysuccinimidyl ester (199 Da) (CT1), or a 6-trimethylammoniumhexanoic acid-N-hydroxysuccinimidyl ester (129 Da) (CT2) is coupled beforehand to the amino function at the 5′-end. The methylated cytosine is detected by the ligation of Seq. ID 17 and Seq. ID 18, while, on the other hand, the unmethylated state, which is represented by a thymine, is detected by the ligation of Seq. ID 18 and Seq. ID 19. Both oligonucleotides hybridize to the complementary strand each time. The oligonucleotides are cleaved by addition of a flap endonuclease (Cleavase VIII endonuclease). The detection of the ligation product is based on the detection of the mass of the remaining nucleotides (CT1+dC or CT2+dC) by means of MALDI-TOF mass spectrometry. The detection limit is decisively reduced due to the size of the products and the defined single negative charge.

### Example 8

Here, a defined fragment of 242 bp length is amplified with the specific primer oligonucleotides TAA GTA TGT TGA AGA AAG ATT ATT GTA G and phosphate-CGC ATC AAC TAA ATC ATT AAA A. This amplificate was digested with lambda-exonuclease, so that the 5′-phosphate-modified strand was digested and removed and only ssDNA (ss=single stranded) was still present. This was now hybridized to an oligonucleotide with the sequence 5′-phosphate-TC TTT CAA AAT TCA CAT-amino-3′ (Seq. ID 20) with the formation of a duplex structure. This oligonucleotide is bound via its 3′-amino modification to an activated surface. Then a ligation mix was added, which contained ligase, ligase buffer and one of the following two oligonucleotides: 5′-DMT-ACT-3′ (Seq. ID 21) or 5′-MMT-ACG-3′ (Seq. ID 22) (DMT=dimethyltrityl, MMT=monomethyltrityl). The methylated cytosine was detected by the ligation of Seq. ID 20 and Seq. ID 21. The trityl group was then cleaved by the addition of acidic matrix and its mass was analyzed in the mass spectrometer. In an unmethylated sample, during the bisulfite reaction, a T was incorporated instead of a C, the sequence ID 22 was ligated and detected by its MMT group. All possible CpG positions in the described ligase reaction can be investigated by the use of additional trityl groups.

1. A method for the detection of nucleic acid sequences is hereby characterized in that the following steps are conducted:
a) at least one nucleic acid is bound to a solid phase;
b) probe molecules are hybridized to the nucleic acids in a sequence-specific manner, whereby the probe molecules are provided with a cleavable bond and a mass label, which is specific for the probe molecule;
c) removal of the unhybridized probe molecules;
d) contacting of the hybridized probe molecules with a matrix, which cleaves said cleavable bonds and at the same time serves as the matrix in a MALDI mass spectrometer;
e) detection of the mass label at those positions where the nucleic acid was bound.

2. The method according to claim 1, further characterized in that the nucleic acid sequences to be detected are DNA sequences and particularly sequences that are variable between different nucleic acids and that contain SNPs, point mutations, deletions, inversions or insertions.

3. The method according to claim 1, further characterized in that the nucleic acid sequences to be detected are chemically pretreated DNA sequences and particularly sequences treated with bisulfite, which serve for the detection of DNA methylation at specific CpG positions.

4. The method according to claim 1, further characterized in that the nucleic acid is amplified prior to binding to the solid phase, whereby this can be preferably produced by means of enzymatic primer extension, PCR, rolling circle amplification, ligase chain reaction or other method.

5. The method according to claim 1, further characterized in that the solid phase is the sample support of a mass spectrometer.

6. The method according to claim 5, further characterized in that the solid-phase surface is comprised of silicon, glass, polystyrene, aluminum, steel, iron, copper, nickel, silver, or gold.

7. The method according to claim 1, further characterized in that the solid phase can be utilized in the sample support of a MALDI mass spectrometer.

8. The method according to claim 1, further characterized in that several nucleic acids are disposed on the solid phase surface in the form of a rectangular or hexagonal grid.

9. The method according to claim 1, further characterized in that the probe molecules are DNA or modified DNA.

10. The method according to claim 1, further characterized in that the probe molecules are LNA, PNA or corresponding hybrids thereof, also with DNA or modified DNA.

11. The method according to claim 9, further characterized in that after the hybridization, the probe molecules are modified enzymatically by primer extension.

12. The method according to claim 9, further characterized in that after the hybridization, the probe molecules are modified enzymatically by ligation.

13. The method according to one of claims 11 or 12, further characterized in that the mass label is first joined with the probe molecule as a consequence of the enzymatic modification.

14. The method according to claim 1, further characterized in that the mass label bears a single positive or a single negative charge.

15. The method according to claim 1, further characterized in that the mass of a label differs each time by at least 1 Da from the masses of all other labels used in one experiment.

16. The method according to claim 3, further characterized in that the probe molecules comprise at least one CG, TG or CA dinucleotide.

17. The method according to claim 3, further characterized in that in step b), the amplificates are hybridized on two classes of probe molecules, each with at least one member, whereby the probe molecules of the first class preferably hybridize to the sequence which arises from the chemical treatment of the genomic DNA, if a cytosine to be investigated was present in the methylated state in the genomic DNA and whereby the probe molecules of the second class preferably hybridize to the sequence which arises from the chemical treatment of the genomic DNA, if a cytosine to be investigated was present in the unimethylated state in the genomic DNA.

18. The method according to claim 3, further characterized in that in step b), a hybridization is produced on two classes of probe molecules, each with at least one member, whereby the probe molecules of the first class preferably hybridize to the sequence which arises from the chemical treatment of the genomic DNA, if a cytosine to be investigated was present in the methylated state in the genomic DNA and less preferably to the sequence which arises from the chemical treatment of the genomic DNA, if a cytosine to be investigated was present in the unmethylated state in the genomic DNA, and whereby the oligomers of the second class hybridize to the amplificate to be investigated essentially independently of the methylation of said specific cytosine in the genomic DNA.

19. The method according to claim 3, further characterized in that in step b), a hybridization is produced on two classes of probe molecules, each with at least one member, whereby the probe molecules of the first class preferably hybridize to the sequence which arises from the chemical treatment of the genomic DNA, if a cytosine to be investigated was present in the unmethylated state in the genomic DNA and less preferably to the sequence which arises from the chemical treatment of the genomic DNA, if a cytosine to be investigated was present in the methylated state in the genomic DNA, and whereby the oligomers of the second class hybridize to the amplificate to be investigated essentially independently of the methylation of said specific cytosine in the genomic DNA.

20. The method according to claim 1, wherein the nucleic acid was obtained from cell lines, blood, sputum, stool, urine, cerebrospinal fluid, tissue embedded in paraffin (for example, tissue from eyes, intestine, kidney, brain, heart, prostate, lung, breast or liver), histological slides or all possible combinations thereof.

21. Use of a method according to claim 1 for the diagnosis and/or prognosis of adverse events for patients or individuals, whereby these adverse events belong to at least one of the following categories: undesired drug effects; cancer diseases; CNS malfunctions, damage or disease; symptoms of aggression or behavioral disturbances; clinical, psychological and social consequences of brain damage; psychotic disturbances and personality disorders; dementia and/or associated syndromes; cardiovascular disease, malfunction and damage; malfunction, damage or disease of the gastrointestinal tract; malfunction, damage or disease of the respiratory system; lesion, inflammation, infection, immunity and/or convalescence; malfunction, damage or disease of the body as a consequence of an abnormality in the development process; malfunction, damage or disease of the skin, the muscles, the connective tissue or the bones; endocrine and metabolic malfunction, damage or disease; headaches or sexual malfunction.

22. Use of a method according to claim 1 for distinguishing cell types or tissues or for investigating cell differentiation.

23. A kit, comprising a solid phase for immobilizing the nucleic acid, probe molecules, as well as components for conducting the mass-spectrometric measurement as well as instructions for conducting a method according to claim 1.
